(19) United States
(12) Patent Application Publication
(10) Pub. No.: US 2026/0294499 A1
(43) Pub. Date: Oct. 1, 2026

(54) CARDIAC ABLATION PLANNING AND RESTRICTION OF ABLATION TO ELECTRODES IN PROXIMITY TO THE PLANNED ABLATION ZONE

(71) Applicant: Boston Scientific Scimed, Inc., Maple Grove, MN (US)

(72) Inventor: Brendan Early KOOP, Ham Lake, MN (US)

(21) Appl. No.: 19/578,040

(22) Filed: Mar. 25, 2026

Related U.S. Application Data
(60) Provisional application No. 63/777,504, filed on Mar. 25, 2025.

(51) Int. Cl. A61B 18/00; A61B 34/10

(52) U.S. Cl. CPC A61B 18/00; A61B 34/10; A61B 2018/0016; A61B 2018/00267; A61B 2018/00351; A61B 2018/00577; A61B 2018/00613; A61B 2018/00702; A61B 2018/00875; A61B 2018/00904; A61B 2034/107

(57) ABSTRACT

A system to perform electroporation ablation of a tissue in a chamber of a patient’s heart is disclosed. The system includes a catheter having an electrode assembly having ablation electrodes, wherein the catheter is adapted to position the electrode assembly in proximity with the tissue. The system also includes a controller to receive a planned ablation zone on an electroanatomical map of the heart. The planned ablation zone is representative of target tissue on a surface of the tissue intended for ablation. The controller identifies, from among the ablation electrodes, a first subset of ablation electrodes that are within a predetermined proximity to the planned ablation zone and a second subset of ablation electrodes that are not within the predetermined proximity to the planned ablation zone. The controller applies a pulsed electrical signal to the first subset of ablation electrodes and not to the second subset of ablation electrodes.

### CROSS REFERENCE TO RELATED APPLICATIONS

The present application claims priority to U.S. Provisional Patent Application No. 63/777,504, filed Mar. 25, 2025, the disclosure of which is incorporated herein in its entirety.

### TECHNICAL FIELD

The present disclosure relates to medical systems and methods for ablating tissue in a patient. More specifically, the present disclosure relates to medical systems and methods for ablation of tissue by electroporation.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electrical field is applied to cells to increase the permeability of the cell membrane. The electroporation can be reversible or irreversible, depending on the strength of the electric field. If the electroporation is reversible, the increased permeability of the cell membrane can be used to introduce chemicals, drugs, and/or deoxyribonucleic acid (DNA) into the cell, prior to the cell healing and recovering. If the electroporation is irreversible, the affected cells are killed through apoptosis.

Irreversible electroporation can be used as a nonthermal ablation technique. In irreversible electroporation, trains of short, high voltage pulses are used to generate electric fields that are strong enough to kill cells through apoptosis. In ablation of cardiac tissue, irreversible electroporation can be a safe and effective alternative to the indiscriminate killing of thermal ablation techniques, such as RF ablation and cryoablation. Irreversible electroporation can be used to kill targeted tissue, such as myocardium tissue, by using an electric field strength and duration that kills the targeted tissue but does not permanently damage other cells or tissue, such as non-targeted myocardium tissue, red blood cells, vascular smooth muscle tissue, endothelium tissue, and nerve cells. There is a continuing need for improved devices and methods for performing cardiac tissue ablation through irreversible electroporation.

### SUMMARY

In Example 1, a system to perform electroporation ablation of a tissue in a chamber of a patient’s heart, the system comprising: a catheter including an electrode assembly having a plurality of ablation electrodes, wherein the catheter is adapted to position the electrode assembly in proximity with the tissue; and a controller configured to: receive a planned ablation zone on an electroanatomical map of the heart, the planned ablation zone representative of a target tissue on a surface of the tissue intended for ablation; identify, from among the plurality of ablation electrodes, a first subset of ablation electrodes that are within a predetermined proximity to the planned ablation zone, and a second subset of ablation electrodes that are not within the predetermined proximity to the planned ablation zone; and apply a pulsed electrical signal to the first subset of ablation electrodes and not to the second subset of ablation electrodes.

In Example 2, the system of Example 1, wherein the planned ablation zone is received via user input.

In Example 3 the system of Example 2, wherein the planned ablation zone is user-selected from an automatically generated suggested planned ablation zone.

In Example 4, the system of Example 3, wherein the suggested planned ablation zone is generated based on an identified cardiac procedure.

In Example 5, the system of any of Examples 1-4, wherein the controller is configured to determine ablation electrodes in contact with the tissue and ablation electrodes within the predetermined proximity to the planned ablation zone.

In Example 6, the system of Example 5, wherein the controller is configured to determine ablation electrodes determined in contact with the tissue within the planned ablation zone.

In Example 7, the system of any of Examples 1-6, wherein the controller is configured to determine electrodes within the predetermined proximity to the tissue via an impedance measurement.

In Example 8, the system of Example 7, wherein the controller is configured to determine the impedance measurement via a pre-pulse signal.

In Example 9, the system of Example 8, wherein the controller is configured to apply the pre-pulse signal to the ablation electrodes only.

In Example 10, the system of any of Examples 1-10, wherein the controller is further configured to generate a visualization of the planned ablation zone with respect to the tissue.

In Example 11, the system of Example 10, wherein the controller is configured to generate a representation of the electrode assembly of the catheter with respect to the planned ablation zone.

In Example 12, the system of Example 11, wherein the controller is configured to generate a visualization identifying the first subset of ablation electrodes.

In Example 13, the system of Example 12, wherein the visualization further identifies the second subset of ablation electrodes.

In Example 14, the system of any of Examples 1-13, wherein the first subset of ablation electrodes includes only ablation electrodes in contact with the target tissue and within the planned ablation zone and the second subset of ablation electrodes includes ablation electrodes not in contact with the tissue or ablation electrodes not within the planned ablation zone.

In Example 15, the system of any of Examples 1-14, wherein the controller is further is configured to adjust ablation parameters based on the identified first subset of ablation electrodes.

In Example 16, a system to perform electroporation ablation of a tissue in a chamber of a patient’s heart, the system comprising: a catheter including an electrode assembly having a plurality of ablation electrodes, wherein the catheter is adapted to position the electrode assembly in proximity with the tissue; and a controller configured to: receive a planned ablation zone on an electroanatomical map of the heart, the planned ablation zone representative of a target tissue on a surface of the tissue intended for ablation; identify, from among the plurality of ablation electrodes, a first subset of ablation electrodes that are within a predetermined proximity to the planned ablation zone, and a second subset of ablation electrodes that are not within the predetermined proximity to the planned ablation zone; and apply a pulsed electrical signal to the first subset of ablation electrodes and not to the second subset of ablation electrodes.

In Example 17, the system of Example 16, wherein the planned ablation zone is received via user input.

In Example 18, the system of Example 17, wherein the planned ablation zone is user-selected from an automatically generated suggested planned ablation zone.

In Example 19, the system of Example 18, wherein the suggested planned ablation zone is generated based on an identified cardiac procedure.

In Example 20, the system of Example 16, wherein the controller is configured to determine ablation electrodes in contact with the tissue and ablation electrodes within the predetermined proximity to the planned ablation zone.

In Example 21, the system of Example 20, wherein the controller is configured to determine ablation electrodes determined in contact with the tissue within the planned ablation zone.

In Example 22, the system of Example 16, wherein the controller is configured to determine electrodes within the predetermined proximity to the tissue via an impedance measurement.

In Example 23, the system of Example 22, wherein the controller is configured to determine the impedance measurement via a pre-pulse signal.

In Example 24, the system of Example 23, wherein the controller is configured to apply the pre-pulse signal to the ablation electrodes only.

In Example 25, the system of Example 16, wherein the controller is further configured to generate a visualization of the planned ablation zone with respect to the tissue.

In Example 26, the system of Example 25, wherein the controller is configured to generate a representation of the electrode assembly of the catheter with respect to the planned ablation zone.

In Example 27, the system of Example 26, wherein the controller is configured to generate a visualization identifying the first subset of ablation electrodes.

In Example 28, the system of Example 27, wherein the visualization further identifies the second subset of ablation electrodes.

In Example 29, the system of Example 16, wherein the first subset of ablation electrodes includes only ablation electrodes in contact with the target tissue and within the planned ablation zone and the second subset of ablation electrodes includes ablation electrodes not in contact with the tissue or ablation electrodes not within the planned ablation zone.

In Example 30, the system of Example 16, wherein the controller is further is configured to adjust ablation parameters based on the identified first subset of ablation electrodes.

In Example 31, a system to perform electroporation ablation of a tissue in a chamber of a patient’s heart, the system comprising:

a catheter including an electrode assembly having a plurality of ablation electrodes, wherein the catheter is adapted to position the electrode assembly in proximity with the tissue; and a controller configured to: receive a planned ablation zone on an electroanatomical map of the heart, the planned ablation zone representative of a target tissue on a surface of the tissue intended for ablation; identify, from among the plurality of ablation electrodes, a first subset of ablation electrodes that are in contact with the target tissue within a predetermined proximity to the planned ablation zone, and a second subset of ablation electrodes that are not in contact with the target tissue within the predetermined proximity to the planned ablation zone; apply a pulsed electrical signal to the first subset of ablation electrodes and not to the second subset of ablation electrodes; and generate a visualization of the planned ablation zone with respect to the tissue.

In Example 32, the system of Example 31, wherein the controller is configured to identify the first and second subsets of ablation electrodes via a pre-pulse signal.

In Example 33, the system of Example 32, wherein the controller is configured to determine electrodes in contact with the tissue via an impedance measurement.

In Example 34, a method of performing electroporation ablation of a tissue in a chamber of a patient’s heart, comprising a catheter including an electrode assembly having a plurality of ablation electrodes, wherein the catheter is adapted to position the electrode assembly in proximity with the tissue, the method comprising: receiving a planned ablation zone on an electroanatomical map of the heart, the planned ablation zone representative of a target tissue on a surface of the tissue intended for ablation; identifying, from among the plurality of ablation electrodes, a first subset of ablation electrodes that are within a predetermined proximity to the planned ablation zone, and a second subset of ablation electrodes that are not within the predetermined proximity to the planned ablation zone; and applying a pulsed electrical signal to the first subset of ablation electrodes and not to the second subset of ablation electrodes.

In Example 35, the method of Example 34, wherein the first set of ablation electrodes that are within the predetermined proximity to the planned ablation zone include ablation electrodes that are in contact target tissue and within the planned ablation zone.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system.

FIGS. 2A and 2B are perspective and end view illustrations, respectively, of a distal portion of a splined catheter for use in the electrophysiology system of FIG. 1.

FIG. 3 is a block diagram illustrating an embodiment of a controller for use with the example electrophysiology system of FIG. 1.

FIG. 4 is a block diagram illustrating an embodiment of a configuration of the controller of FIG. 3.

FIG. 5 is a block diagram illustrating another embodiment of a configuration of the controller of FIG. 3.

FIGS. 6A-6D are schematic diagrams illustrating visualizations generated with the configuration of the controller of FIG. 4 during a cardiac procedure with the electrophysiology system of FIG. 1.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

As applied to electrophysiology systems, irreversible electroporation uses high voltage, short pulses to kill cells such as myocardium through apoptosis while sparing other adjacent tissues including the esophageal vascular smooth muscle and endothelium. Irreversible electroporation treatment can be delivered in multiple therapy sections. A therapy section, which may have a duration on the order of milliseconds, may include a plurality of electrical pulses, such as a few dozen pulses, generated and delivered by an electroporation device, which is powered by an electroporation generator, to generate an electric field of sufficient strength to create transmural lesions. In one example, one or more catheters may be advanced in a minimally invasive fashion through vasculature to a target location, such as in the heart. The methods described here may include introducing a device into an endocardial space of the heart. A pulse waveform may be generated and delivered to electrodes of the device to ablate tissue.

Some electroporation devices can include catheters having a three-dimensional electrode array, such ablation electrodes disposed on spline, baskets, loops or balloons. In such a configuration, some ablation electrodes can be contact with the target tissue and other ablation electrodes are not in contact with the target tissue during the ablation procedure. For instance, ablation electrodes not in contact with the target tissue during ablation procedures can be disposed in a blood pool in a cardiac cavity. Activation of ablation electrodes not in contact with the target tissue during ablation can cause adverse effects among which can include bubble formation and skeletal muscle stimulation. Additionally, activation of ablation electrodes floating in blood can cause hemolysis with potential downstream effects including acute kidney injury. The procedures were also time-consuming and required significant physician decision-making regarding ablation parameters, timing, and catheter positioning. Maintaining consistent catheter position is a challenge due to cardiac motion, which can lead to failed procedures when the catheter moves significantly between pulse trains.

Typical approaches to therapy via electroporation include a clinician manually positioning the catheter, deciding parameters for each ablation, and repeating this process multiple times to form complete ablation lines or lesions. The manual process is prone to errors due to the number of decisions and limited time for treatment during cardiac procedures. Also, fixed timing between ablations did not account for cardiac motion or varying tissue contact. Additionally, gaps between lesions could develop if the spacing and timing of sequential ablations were not carefully controlled.

The disclosure is directed to a system for use in a clinic setting such as for an electrophysiological procedure. The system includes or is configured to employ a catheter having ablation electrodes and, in some embodiments, mapping electrodes. For example, the catheter includes ablation electrodes disposed on a three-dimensional electrode array distal to an elongate shaft. The catheter is coupled to a controller. In one embodiment, the controller is configured to track the catheter in the cardiac tissue and generate a representation of the catheter with respect to cardiac tissue on a display. In embodiments, the controller tracks the catheter with respect to the cardiac tissue in real time. The controller is configured to receive a planned ablation zone on an anatomical map of the heart, in which the planned ablation zone representative of target tissue such as tissue to be targeted during the procedure or an area on a surface of the tissue intended for ablation. The controller is configured to determine a first subset of ablation electrodes within a predetermined proximity of the planned ablation zone. In one embodiment, the first subset of ablation electrodes includes ablation electrodes in contact with tissue within the planned ablation zone. In some embodiments, the first subset of ablation electrodes is fewer than the plurality of ablation electrodes. In some embodiments, the controller is also configured to determine which of the ablation electrodes, if any, are in a second subset of ablation electrodes are not within the predetermined proximity to the planned ablation zone. In some embodiments, the second subset of ablation electrodes are not in contact with the target tissue or not in the planned ablation. In some embodiments, the controller is configured to present on the anatomical map or otherwise on the display the determined first subset of ablation electrodes. In one embodiment, the controller is configured to present on the anatomical map the ablation electrodes in contact with tissue corresponding with the planned ablation zone, such as in real time. A pulsed electrical signal configured for ablation via electroporation is applied to the first subset of ablation electrodes and not to the second subset of ablation electrodes. The system may be configured to employ a range of tissue proximity assessment processes or tissue contact assessment processes to determine whether an ablation electrode is within the predetermined proximity to the tissue represented by the planned ablation zone. In one embodiment utilizing impedance-based proximity or contact assessment processes in which impedance between a given ablation electrode and a second electrode (e.g., another ablation electrode, a sensing electrode, or a remote indifferent electrode) is utilized as the tissue proximity parameter.

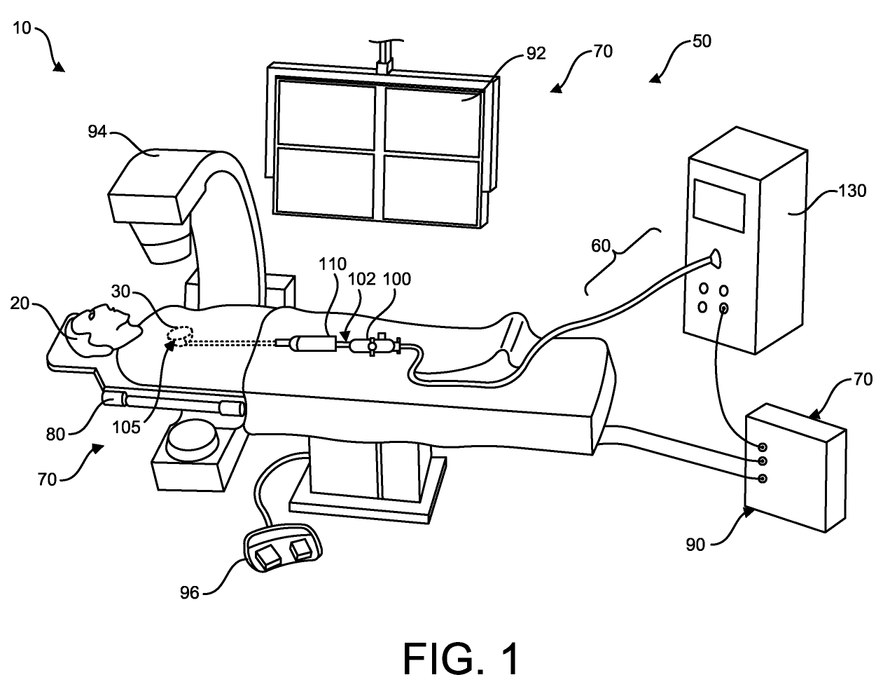

FIG. 1 illustrates an example clinical setting 10 for treating a patient 20, such as for treating a heart 30 of the patient 20, using an electrophysiology system 50, in accordance with the disclosure. The electrophysiology system 50 includes an electroporation catheter system 60 and an electro-anatomical mapping (EAM) system 70. The example electroporation catheter system 60 includes an electroporation catheter 105, an introducer sheath 110, and an electroporation console 130. Additionally, the electroporation catheter system 60 includes various connecting elements, such as cables and tubing, that operably connect the components of the electroporation catheter system 60 to one another and to the components of the EAM system 70. Also, the clinical setting 10 can include additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1.

The electroporation catheter system 60 is configured to deliver ablation electric field energy to targeted tissue in the patient’s heart 30 to create cell death in tissue, for example, rendering the tissue incapable of conducting electrical signals. Also, the electroporation catheter system 60 is configured to generate electric fields using the electroporation catheter 105 to create and present, on the display 92, an electro-anatomical map of the patient’s heart to aid a clinician in planning ablation by irreversible electroporation using the electroporation catheter 105 prior to delivering ablation electric field energy. In embodiments, the electroporation catheter system 60 is configured to generate the electric fields based on characteristics of the electroporation catheter 105 and the position of the electroporation catheter 105 in the patient 20, such as in the heart 30 of the patient 20. The electroporation catheter system 60 is configured to generate graphical representations of the electroporation catheter and the electro-anatomical map based on characteristics of the electroporation catheter 105 and the position of the electroporation catheter 105 in the patient 20, such as in the heart 30 of the patient 20, and the characteristics of the tissue surrounding the catheter 105, such as measured impedances of the tissue. The electroporation catheter system 60 can include additional features.

The introducer sheath 110 is operable to provide a delivery conduit through which the electroporation catheter 105 can be deployed to the specific target sites within the patient’s heart 30. Access to the patient’s heart can be obtained through a vessel, such as a peripheral artery or vein often in the groin or possibly in the shoulder or neck. Once access to the vessel is obtained, the electroporation catheter 105 can be navigated to within the patient’s heart, such as within a chamber of the heart.

In one example, the electroporation catheter 105 is a mapping and ablation catheter, which can be deployed in mapping procedures in cooperation with the EAM system 70 as well as to deliver ablation electric field energy and ablate tissue via irreversible electroporation. The example electroporation catheter 105 includes an elongated catheter shaft and distal end region configured to be deployed proximate target tissue, such as within a chamber of the patient’s heart or the wall of a pulmonary vein ostium. The shaft can extend from an access point in the patient to the target tissue and generally defines a longitudinal axis of the electroporation catheter 105. A proximal end region of the catheter can include a handle having user manipulatable controls for the catheter 105. The distal end region may include a basket, balloon, spline, loop, configured tip, or other electrode deployment mechanism coupled to the shaft. The electrode deployment mechanism includes an electrode assembly, or array, comprising an electrode. For example, the electrode assembly can include a plurality of spaced-apart electrodes or multiple spaced-apart sets or groups of spaced-apart electrodes. In some examples, an electrode, such as a plurality of spaced-apart electrodes, can be deployed on the catheter shaft in addition to or instead of an electrode on the electrode deployment mechanism. For instance, the electrode deployment mechanism includes a plurality of flexible support members configured to form a basket, and at least a some of the electrodes are disposed on the flexible support members.

The electroporation catheter 105 is configurable in a plurality of states. For example, when the distal end region of the catheter 105 is within a sheath as a catheter assembly, such as to travel to the patient to the chamber of the heart, the electrode deployment mechanism and electrode assembly are in a collapsed state to fit within the sheath. Once the catheter has reached the destination in the chamber of the heart, for example, the sheath is retracted from the distal region of the catheter 105 (or the shaft catheter is extended past the sheath) and the electrode deployment mechanism and electrode assembly can be arranged in an expanded state. The electrode assembly has a collapsed shape when the catheter 105 is in the collapsed state and an expanded shape when the catheter 105 is in the expanded state. In some examples, the electrode assembly has more than two states.

In one example, the plurality of electrodes can be formed of a conductive, solid-surface, biocompatible material and are spaced-apart across insulators. Each of the plurality of electrodes is electrically coupled to a corresponding elongated lead conductor that extend along the shaft to a catheter proximal end. In one example, each electrode of the spaced-apart electrodes corresponds with a separate, single lead conductor. In another example, a plurality of electrodes may be coupled to a single lead conductor. Other configurations are contemplated. The plurality of lead conductors can be insulated from one another within an insulating sheath along the catheter shaft, such as with an insulating polymer sheath. The lead conductors can be electrically coupled to plug in the proximal region of the electroporation catheter 105, such as a plug configured to be mechanically and electrically coupled to the electroporation console 130, for example, either directly or via intermediary electrical conductors such as cabling.

The electroporation console 130 includes a controller, such one or more controllers, processors, or computers, that executes instructions or code, such as processor-executable instructions, out of a non-transitory computer readable medium, such as a memory device, or memory, to cause, such as control or perform, the aspects of the electroporation catheter system 60. In one example, the electroporation console 130 is configured to provide an electrical signal, such as a plurality of concurrent or space-apart-time electrical signals, to the electrically connected electroporation catheter 105 along lead conductors to the spaced-apart electrodes. The spaced-apart electrodes are configured to generate a selected electrical field proximate the target tissue, based on the electrical signals from the electroporation console 130, such as to effect ablation.

The electroporation console 130 can generate electrical signals and select which electrodes in the electrode array will receive the electrical signals. A first electrode, or first group of electrodes, can be selected to be an anode and a different, second electrode, or second group of electrodes, can be selected to be a cathode, such that electrical fields can be generated between the anode and cathode based on signals, such as pulses, provided to the electrodes from the electroporation console 130. The console 130 provides electric pulses of different lengths and magnitudes to the electrodes on the catheter 105. The electric pulses can be provided in a continuous stream of pulses or in multiple, separate trains of pulses. Pulse parameters of interest include the number of pulses, the duty cycle of the pulses, the spacing of pulse trains, the voltage or magnitude of the pulses including the peak voltages, and the duration of the voltages. For example, the console 130 can select two or more electrodes of the electrode assembly and provides pulses to the selected electrodes to generate electric fields between the selected electrodes.

In an ablation mode, the console can select electrodes to provide pulsed field ablation (PFA). For example, PFA can be performed with monophasic waveforms and biphasic waveforms. Without being bound to a particular theory, electric field strengths in the range of generally 200-250 volts per centimeter (V/cm) with microsecond-scale pulse duration have been demonstrated to provide reversible electroporation in cardiac tissue. Electric field strengths at approximately 400 V/cm have been demonstrated to provide irreversible electroporation in cardiac tissue of interest, such as targeted myocardium tissue and endocardium tissue, with demonstrable sparing of red blood cells, vascular smooth muscle tissue, endothelium tissue, nerves and other non-targeted proximate tissue.

Additionally, the electrode assembly on catheter 105 can be operated in a selected mode such as monopolar mode or bipolar mode. During monopolar operation of the catheter 105, an electrode, a group of electrodes, or the entire electrode assembly are configured as one of an anode or a cathode. None of the electrodes in the electrode assembly are configured as a the other of the cathode or the anode. Instead, the other of the cathode or the anode is provided in the form of a pad dispersive electrode located on the patient, typically on the back, buttocks, or other suitable anatomical location during electroporation. An electrical field is formed between an activated electrode of the electrode assembly and the pad dispersive electrode. In an alternative configuration, a return electrode, such as a plurality of return electrodes, can be disposed on the shaft of the catheter. An electrical field is formed between an activated electrode of the electrode assembly and the return electrode on the shaft of the catheter 105. During bipolar operation of the catheter 105, a first set of one or more electrodes of the electrode assembly, is configured as the anode and a second set of one or more electrodes of the electrode assembly, is configured as the cathode, to generate the electric field. In this example, a pad dispersive electrode is not used, and the electrical field is not extended in the patient’s body, but rather through a localized portion of tissue proximate the electrode assembly.

In the illustrated examples, the catheter 105 is a mapping and ablation catheter, and the electrodes can include ablation electrodes that are configured to deliver ablation electric field energy and mapping electrodes for mapping purposes. In some configurations, the mapping electrodes are configured to be used to collect electrical signals to be used to generate via the operably coupled EAM system 70, and display via the operably coupled display 92, detailed three-dimensional geometric anatomical maps or representations of the cardiac chambers as well as electro-anatomical maps in which cardiac electrical activity of interest is superimposed on the geometric anatomical maps. In some examples, an electrode can operate as an ablation electrode in an ablation mode of the electrophysiology system 50 and as a mapping electrode in a mapping mode of the system 50. Mapping electrodes on the electroporation catheter 105 can measure electrical signals and generate output signals that can be processed by the mapping and navigation controller 90 to generate an electro-anatomical map. In some instances, electro-anatomical maps are generated before ablation for determining the electrical activity of the cardiac tissue within a chamber of interest. In some instances, electro-anatomical maps are generated after ablation in verifying the desired change in electrical activity of the ablated tissue and the chamber. The mapping electrodes may also be used to determine the position of the catheter 105 in three-dimensional space within the body. For example, when the operator moves the distal end of the catheter 105 within a cardiac chamber of interest, the boundaries of catheter movement can be used by the mapping and navigation controller 90 to form the anatomical map of the chamber. The chamber anatomical map may be used to facilitate navigation of the catheter 105 without the use of ionizing radiation such as with fluoroscopy, and for tagging locations of ablations as they are completed in order to guide spacing of ablations and aid the clinician in ablating the anatomy of interest. In some examples, an electrode in the electrode assembly can be configured to only perform an ablation or the electrode in the electrode assembly can be configured to only perform mapping.

The EAM system 70 is configured to generate the electro-anatomical map for display on the display 92. The EAM system 70 is operable to track the location of the various components of the electroporation catheter system 60, and to generate high-fidelity three-dimensional anatomical and electro-anatomical maps of the heart, including portions of the heart such as cardiac chambers of interest or other structures of interest such as the sinoatrial node or atrioventricular node. In one illustrative example, the EAM system 70 can include the OPAL™ HDx mapping system marketed by Boston Scientific Corporation. Also, the mapping and navigation controller 90 of the EAM system 70 includes one or more controllers, such as microprocessors or computers, that execute code out of memory to control or perform functional aspects of the EAM system 70, in which the memory, can be part of the one or more controllers, microprocessors, computers, or part of a memory device accessible through a computer network.

The EAM system 70 generates a localization field, via the field generator 80, to define a localization volume about the heart 30, and a location sensor or sensing element on a tracked device, such as sensors on the electroporation catheter 105, generate an output that can be processed by the mapping and navigation controller 90 to track the location of the sensor, and consequently, the corresponding device, within the localization volume. In the illustrated example, the device tracking is accomplished using magnetic tracking techniques, in which the field generator 80 is a magnetic field generator that generates a magnetic field defining the localization volume, and location sensors on the tracked devices are magnetic field sensors.

In other examples, impedance tracking methodologies may be employed to track the locations of the various devices. In such examples, the localization field is an electric field generated, for example, by an external field generator arrangement, such as surface electrodes, by intra-body or intra-cardiac devices, such as an intracardiac catheter, or both. In these examples, the location sensing elements can constitute electrodes on the tracked devices that generate outputs received and processed by the mapping and navigation controller 90 to track the location of the various location sensing electrodes within the localization volume.

The EAM system 70 can be equipped for both magnetic and impedance tracking capabilities. In such examples, impedance tracking accuracy can, in some instances be enhanced by first creating a map of the electric field induced by the electric field generator within the cardiac chamber of interest using a probe equipped with a magnetic location sensor, as is possible using the OPAL HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the electroporation catheter 105 or another catheter or probe equipped with sensing electrodes, to generate, and display via the display 92, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map or the electro-anatomical map.

The electroporation catheter system 60 can be combined or integrated with the EAM system 70 to allow graphical representations of the electric fields that can be produced by the electroporation catheter 105 to be visualized on an electro-anatomical map of the patient’s heart. The integrated system can include the capability to enhance the efficiency of clinical workflows, including enhancement of providing a visual representation to the clinician of ablation lesions of portions of the patient’s heart created through irreversible electroporation. The integrated system can include generating the graphical representations of the electric fields that can be produced by the electroporation catheter 105, generating the anatomical maps including generating the electro-anatomical maps, and displaying information related to the location and electric field strengths of the electric fields that can be produced by the electroporation catheter 105.

The depiction of the electrophysiology system 50 is intended for illustration or a general overview of the various components of the system 50 and is not intended to imply that the disclosure is limited to any set of components or arrangement of the components. For example, additional hardware components, such as breakout boxes or workstations, can be included in the electrophysiology system 50.

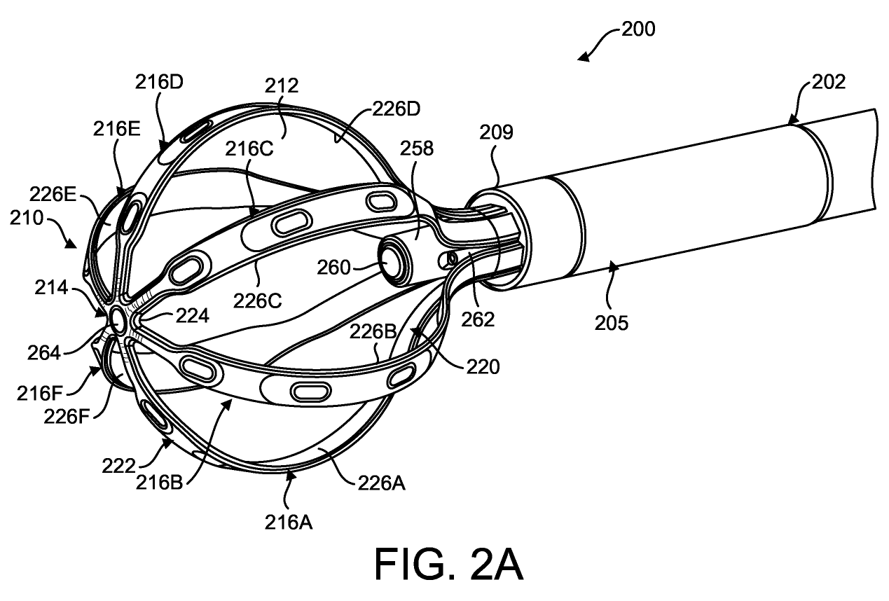

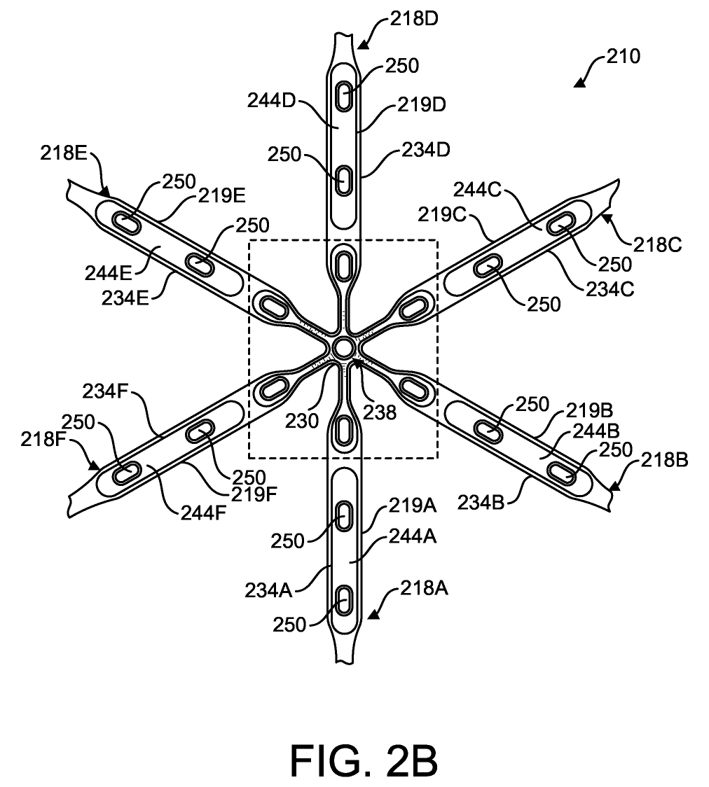

FIGS. 2A and 2B illustrate an example electroporation catheter 200 for mapping ablation of cardiac tissue, which can be an example of catheter 105 and used with the electrophysiology system 50. FIG. 2A is a partial perspective illustration of an electroporation catheter 200 having a catheter distal portion 205. The electroporation catheter 200 has a tubular outer shaft 208 having a shaft distal end 209, and an electrode assembly 210 extending distally from the distal end 209 of the outer shaft 208. In embodiments, the electrode assembly 210 is configured to self-expand from a collapsed state when constrained within a delivery sheath to a pre-defined expanded state defining an inner space, or cavity 212. The electrode assembly includes multiple ablation electrodes configured to receive pulsed electrical signals or waveforms from the electroporation console 130, for creating pulsed electric fields sufficient for ablating target tissue via irreversible electroporation. Additionally, the electrode assembly 210 further includes a plurality of mapping and sensing electrodes configured for, among other things, sensing cardiac electrical signals, localization of the electrode assembly 210 within the patient anatomy such as via the EAM system 70, and determining proximity to target tissue within the anatomy.

FIG. 2B is a partial plan illustration of the electrode assembly 210 of the electroporation catheter 200 in two dimensions to demonstrate the layout of the electrode assembly 210. Referring to FIGS. 2A and 2B together, the electrode assembly 210 has a distally-located central hub portion 214 and a plurality of splines 216A-216F extending proximally from the central hub portion 214. Each respective spline 216A-216F has a distal end portion 217A-217F, a proximal end portion 218A-218F, and an intermediate portion 219A-219F extending between the distal end portion 217A-217F and the proximal end portion 218A-218F. Each of the proximal end portions 218A-218F is attached to and constrained by the distal end 209 of the outer shaft 202.

The splines 216A-216F include a support member 220 and a flexible circuit 222. The flexible circuit 222 is secured to and disposed over an outer surface of the support member 220. The support member 220 includes a support member hub 224 and a plurality of support member branches 226A-226F. The support member 220 provides a structural support of the electrode assembly 210. The support member 220 is formed from a superelastic material, such as a metal or polymer, to provide desired mechanical or structural properties to the electrode assembly 210. In one example, the support member 220 is formed from a superelastic metal alloy such as a nickel-titanium alloy. The flexible circuit 222 includes a flex circuit hub 230 and a plurality of flex circuit branches 234A-234F. In embodiments, the flex circuit hub 230 is disposed over and secured to the support member hub 224. In embodiments, the flex circuit branches 234A-234F are integrally formed with the flex circuit hub 230, and each of the flex circuit branches 234A-234F is disposed over and secured to a respective one of the support member branches 226A-226F.

The flexible circuit 222 includes a distal ablation electrode 238 that has a distal ablation electrode hub portion 240 and a plurality of radial segments 242A-242F. In the example, the distal ablation electrode hub portion 240 is located on the flex circuit hub 230. Additionally, the radial segments 242A-242F formed with the distal ablation electrode hub portion 240. Each of the radial segments 242A-242F extends proximally along a portion of a respective one of the flex circuit branches 234A-234F. The flexible circuit 222 further includes a plurality of proximal ablation electrodes 244A-244F. Each of the proximal ablation electrodes 244A-244F is located on a respective one of the flex circuit branches 234A-234F.

The flexible circuit 222 includes a plurality of spline sensing electrodes 250. In the example, each of the spline sensing electrodes 250 is disposed within a periphery of one of the proximal ablation electrodes 244A-244F or one of the radial segments 242A-242F of the distal ablation electrode 238. For instance, each of the distal-most spline sensing electrode 250 is disposed within a periphery of a respective one of the radial segments 242A-242F of the distal ablation electrode 238 and is electrically isolated from the distal ablation electrode 238. Additionally, a plurality of the more proximally-located spline sensing electrodes 250 is disposed along and within a periphery of a respective one of each of the proximal ablation electrodes 244A-244F and electrically isolated from the proximal ablation electrodes 244A-244F.

In the example, the electroporation catheter 200 includes a central post 258 extending distally from the distal end 209 of the outer shaft 202. The central post 258 extends partially into the cavity 212 and includes a post electrode 260. The post electrode 260 can operate as a reference for unipolar electrograms, in lieu of reliance on surface ECG patch electrodes. An irrigation lumen 261 can be included and supported by the central post 258. The central post 258 may house additional components such as a magnetic navigation sensor. In the illustrated embodiment, the electrode assembly 210 further includes a hub sensing electrode 264 centrally located on the flex circuit hub 230.

The post electrode 260 can provide additional advantages. In one example, the post electrode 260 can operate as a reference for unipolar electrograms, in lieu of reliance on surface ECG patch electrodes as are otherwise known in the art. The location of the post electrode 260 for this purpose positions the reference electrode much closer to the tissue being sensed than is possible with the conventional surface ECG approach, which may advantageously minimize far field noise and provide much sharper unipolar electrograms than what are possible using surface ECG electrodes. The post electrode 260 may also be operable to sense and measure other electrical parameters, e.g., voltages between it and the ablation electrodes or other sensing electrodes on the electrode assembly 210, thereby providing data usable for, in some examples, determining the shape of the electrode assembly during use (including when deformed by forces applied by cardiac walls), and displaying shape information via the EAM system 70.

The hub sensing electrode 264 allows tissue surface mapping to be conducted in a “forward” manner, reducing the need to manipulate the electrode assembly 210 to place the spline sensing electrodes 250 against or proximate the tissue to be mapped. The inclusion of the hub sensing electrodes 264 further enhances bipolar sensing capabilities by providing for, in the illustrated embodiment, six additional bi-poles when paired with any of the distal-most spline sensing electrodes 250.

The example electrode assembly 210 is primarily designed to create relatively localized ablation lesions, or focal lesions, as compared to relatively large diameter circumferential lesions that may be created in pulmonary vein isolation procedures. However, the present disclosure can be readily adapted for a catheter capable of large diameter circumferential lesions. The example electrode assembly 210 can provide a clinician with a wide range of capabilities for monopolar and bipolar focal pulsed field ablation of cardiac tissue, combined with the ability to perform localized, such as at the location of the delivery of pulsed field ablative energy, high fidelity sensing of cardiac tissue, for lesion or conduction block assessment, or tissue contact determinations.

The depiction of the electroporation catheter 200 is intended for illustration or a general overview of components of a catheter for use in system 50 and is not intended to imply that the disclosure is limited to any set of components or arrangement of the components. For example, another electroporation catheter, such as catheters with three-dimensional electrode arrays including basket catheters or ablation-only catheters, can be included in the electrophysiology system 50.

The example of the electroporation catheter 200 includes seven ablation electrodes and nineteen sensing electrodes that can be placed in contact with the target tissue in addition to other electrodes or sensors that are not expected to be placed in contact with the target tissue. The separate ablation electrodes are employed to produce appropriate lesion depths for the treatment of atrial arrythmias. Ablation procedures with the electroporation catheter 200 can be performed in a monopolar mode with an indifferent or patch electrode applied to a patient’s skin, such as skin on the patient’s back. Generally during an ablation procedure, some of the ablation electrodes in the electrode array are in contact with the target tissue while some of the ablation electrodes are not in contact with the target tissue. Rarely, if ever, are all ablation electrodes in contact with the target tissue in such a three-dimensional electrode array. The application of pulsed signals to ablation electrodes not in contact with the target tissue, such as ablation electrodes in the blood pool of the cardiac chamber, e.g., the left atrium, be undesirable for various clinical and/or operational reasons such as microbubble formation, skeletal muscle stimulation, reduced ablation effectiveness, and the like. The electrophysiology system 50 is employed to determine a subset of ablation electrodes in contact with the target tissue, wherein the subset of ablation electrodes is fewer than the plurality of ablation electrodes, and a remainder ablation electrode not in contact with the target tissue and apply a pulsed electrical signal to the subset of ablation electrodes and not to the remainder ablation electrode. In one example, the characteristics of the pulsed electrical signal is configured based on the subset of ablation electrodes.

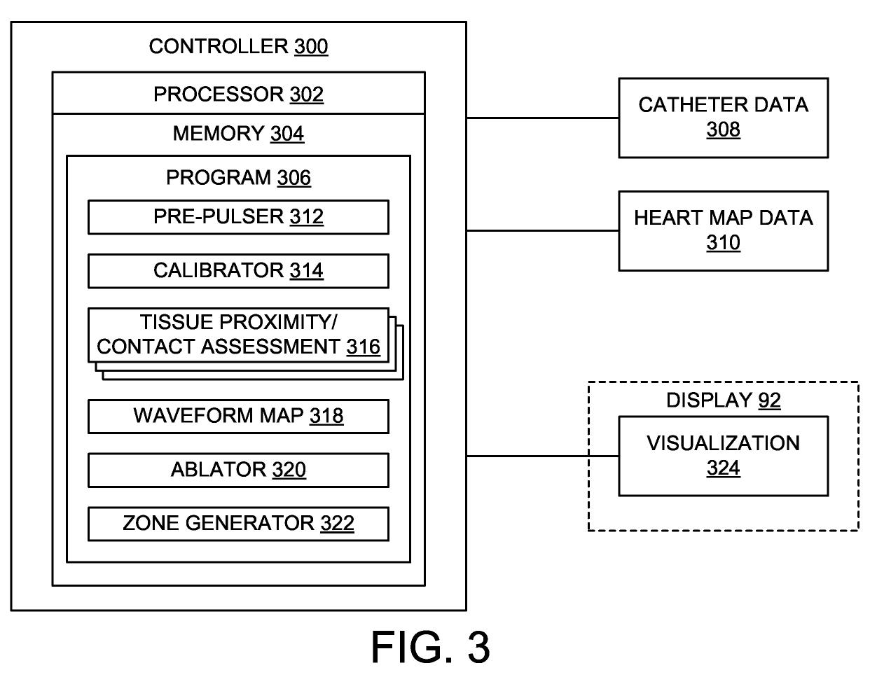

FIG. 3 illustrates an example controller 300 that can be used with the example electrophysiology system 50, such as a controller of the example catheter system 60 or the electroporation console 130. The controller 300 can be implemented to determine which of the ablation electrodes of an electroporation catheter, such as catheter 200, is in contact with target tissue and within a planned ablation zone on the surface of cardiac tissue to be treated and to select or activate such ablation electrodes to provide PFA and deselect or deactivate ablation electrodes that are not in contact with target tissue or not within the planned ablation zone. Further, the controller 300 can be implemented to adapt the PFA waveform provided to the selected ablation electrodes based on the configuration, e.g., the number, spacing, orientation etc. of the selected electrodes. The controller 300 can include a processor 302 and a memory 304. The memory 304 stores processor executable instructions 306. In one example, the processor executable instructions 306 can be in the form of a program, such as a computer program or application. The processor 302 can execute the instructions 306 that can be included in configuring the controller 300. In one example, the controller 300 can be implemented to include a computing device such as a laptop computer, a workstation, a desktop computer, a tablet, or a smartphone or devices such as the electroporation console 130 such as a PFA generator. In such examples, the controller 300 can include additional components such as a display, a touchscreen, speakers or other output devices, a keyboard or other input devices, or communication circuitry such as computer network adapters. The controller 300 may be implemented in a variety of architectures and components, such as the processor 302 and memory 304, may be distributed in various locations.

In one example, the processor 302 may include a plurality of main processing cores to run an operating system and perform general-purpose tasks on an integrated circuit. The processor 302 may also include built-in logic or a programmable functional unit, also on the same integrated circuit. In additional to multiple general-purpose, main processing cores and the application processing unit, controller 300 can include other devices or circuits such as graphics processing units or neural network processing units with the main processing cores. For example, the controller 300 may be used to perform other tasks.

Memory 304 is an example of computer storage media. Computer storage media includes RAM, ROM, EEPROM, flash memory or other memory technology, CD-ROM, digital versatile discs (DVD) or other optical storage, magnetic cassettes, magnetic tape, magnetic disk storage or other magnetic storage devices, USB flash drive, flash memory card, or other flash storage devices, or other storage medium that can be used to store the desired information and that can be accessed by the processor 302. Any such computer storage media may be part of the controller 300 and implemented as memory 304. Memory 304 is a non-transitory, processor readable memory device. Accordingly, a propagating signal by itself does not qualify as storage media or memory 304.

The controller 300 may be configured to receive inputs or information from the electrophysiology system 50, such as inputs from the electroporation catheter system 60 and EAM system 70 including the electroporation console 130 and the mapping and navigation controller 90, for storage in memory 304 and use by the instructions 306. In some embodiments, the controller receives inputs representative of data obtained with the catheter system 60, such as data determined from electrical or physiological signals received from electrodes on the catheter assembly such as the ablation electrodes and sensing electrodes, and tracking devices, and other data, or catheter obtained data 308. Catheter obtained data 308 can include data determined from physiological signals of the heart, electrode location information, tissue proximity information, force or contact information, catheter tip or tissue temperature, acoustic information, catheter electrical coupling information, catheter deployment shape information, electrode properties, respiration phase, blood pressure, impedance information and other physiological information. In some embodiments, catheter obtained data 308 can include or be supplemented with other information collected from sensors during mapping of the heart. In some embodiments, the controller 300 receives an input representative of the anatomical map of the heart, or heart map data 310, which collected heart map data 310 can include the data regarding representations of the geometric anatomical map of the heart and the electro-anatomical map of the heart, such as from the EAM system 70. Heart map data 310 can include data previously collected, such as in the same procedure using a different map dataset, or with a different modality such as computerized tomography (CT), magnetic resonance imaging (MRI), ultrasound, or rotational angiography, and registered to the catheter locating system. Additionally, heart map data 310 can include annotations, markings, or user-added tags of the associated with the anatomical map of the heart that may include markings of anatomical locations of interest or other data to generate visualizations a clinician may find of interest during a procedure.

The program 306 can include a suite of modules stored in memory 304. For instance, the controller 300, via the program 306, can be configured to generate electrical signals with a pre-pulser 312 to provide to the catheter 105, and receive feedback from anatomy as part of catheter obtained data 308. The pre-pulser 312, via the electroporation console 130, can provide electrical signals to the ablation electrodes only in some embodiments and configured in a bipolar mode to determine impedance or current readings from feedback signals. In some embodiments, the pre-pulser 312, via the electroporation console 130, can provide electrical signals to the ablation electrodes and the sensing electrodes configured in a bipolar mode to determine impedance or current readings from feedback signals. The pre-pulse electrical signal can be a continuous signal that is selectively read or can be a selectively applied, discrete pre-pulse signal that is read in response to being applied. The program 306 can includes a calibrator 314 to obtain a baseline measurement, such as readings when the catheter is not in contact with tissue that can be compared to readings when the catheter is in contact with tissue to determine which of the ablation electrodes are in contact with tissue. In one example, the calibrator 314 can be operative with the pre-pulser 312 to provide, for instance, a first pre-pulse signal with the catheter not in contact with the target tissue and a second pre-pulse signal with the catheter in contact with the target tissue, such as with a selectively applied, discrete pre-pulse signal. Additionally, the program 306 can include one or more tissue contact assessment processes 316 that can be applied to determine which of the ablation electrodes are in contact with the target tissue. In the case of a plurality of tissue contact assessment processes 316, and the controller 300 is further configured to select a tissue contact assessment process from the plurality of tissue contact assessment processes. The memory 304 can also include an electrode-waveform map 318, which can be used to determine a corresponding pulsed electrical signal to be applied to the ablation electrodes in contact with the tissue and within the planned ablation zone based on the subset of the ablation electrodes in contact with the tissue. In one example, the electrode-waveform map 318 may be a lookup table, or a set of lookup tables stored in memory 304, although other configurations are contemplated. The program 306 can include an ablator 320 to apply the determine corresponding pulsed electrical signal to the subset of ablation electrodes to effect ablation by electroporation. Still further, the program 306 can include a zone generator 322 for use with the heart map data 310 and the anatomical map of the heart. The zone generator 322 is configured to receive a user input of the planned ablation zone, which is a user-selected area on the anatomical map of the heart as a target of ablation, associate the planned ablation zone, or user-selected area, with the heart map data 310 as an annotation of a target of ablation, and to associate the area on the heart map with a corresponding area of the patient’s heart. In some embodiments, the program is included in as part of an EAM package. In some embodiments, the program includes other tools such as a lesion predictor to indicate the likely region of an lesion created with determined pulse signal parameters, such as on the anatomical map of the heart and prior to the activation of ablation energy to an electrode also tools such as an ablation tag to indicate the region of a lesion resulting from the application of ablation energy, such as on the anatomical map of the heart and subsequent to the activation of ablation energy.

The controller 300 is configured to generate a visualization 324 that can include determined location of the catheter assembly as a single unit based on the received tracking data of the catheter elements with reference to the anatomical map of the heart. The visualization 324 in some embodiments includes a representation of the planned ablation zone on the anatomical map of the heart. In some embodiments, a user is able to input the planned ablation zone with an input device using the visualization as a graphical user interface. In examples, the input device can include a mouse, trackpad, or touchpad screen for manipulating a cursor on the graphical user interface to select a region of the representation of heart tissue as the planned ablation zone corresponding with an area on a surface of the cardiac tissue intended for ablation

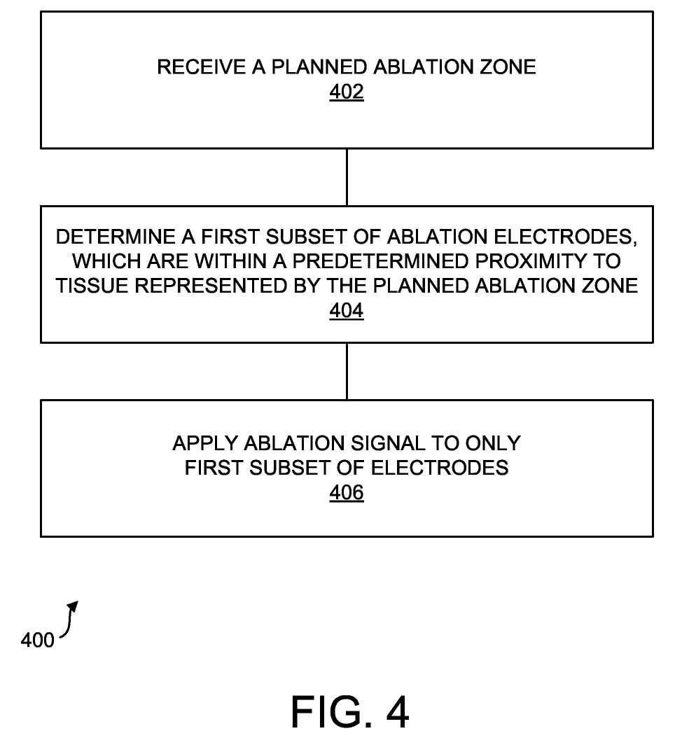

FIG. 4 illustrates a process 400 of configuring a controller, such as controller 300, to receive the planned ablation zone on the anatomical map of the heart, the planned ablation zone representative of an area on a surface of the tissue intended for ablation, identify which of the ablation electrodes of an electroporation catheter, such as catheter 200, are in contact with target tissue and within the planned ablation zone when in use, and to select or activate such ablation electrodes to provide PFA and deselect or deactivate ablation electrodes that are not in contact with target tissue or within the planned ablation zone. Further, the controller 300 can be configured to adapt the PFA waveform provided to the selected ablation electrodes based on the configuration, e.g., number, spacing, orientation, etc. of the selected electrodes. For example, the controller can be implemented as part of the electroporation catheter system 60 or operably coupled to the EAM system 70. Process 400 can be use with a catheter including an electrode assembly having a plurality of ablation electrodes, wherein the catheter is adapted to position the electrode assembly in contact with cardiac tissue in an area on the surface of the tissue designated by user as intended for ablation.

The controller is configured to receive a planned ablation zone on an anatomical map of the heart, in which the planned ablation zone is representative of an area on a surface of the tissue intended for ablation at 402. In some embodiments, the controller is configured to receive data characterizing a planned ablation zone on an electroanatomical map of a heart. For example, the planned ablation zone is provided via a user-selected input to the controller, and the data can be generated via an input device. Embodiments of input devices include devices such as a keyboard, pointing devices (e.g., mouse, trackpad, or stylus), touch input devices (e.g., touchscreen), and voice input devices. In one embodiment, an area of cardiac tissue is presented on a graphical user interface on a display device, and the user can select an area on the representation of cardiac tissue via a mouse or stylus. In one embodiment, the planned ablation zone is user selected in a first instance, such as the area of cardiac tissue is presented on the graphical user interface and the user draws or demarks the planned ablation on the graphical user interface via a stylus, such as a circle or an oval, to correspond with cardiac tissue on the patient. In another embodiment, the graphical user interface presents one or more suggested planned ablation zones on the graphical user interface that correspond with cardiac tissue on the patient, and the user selects from the suggested planned ablation zones or another planned ablation zone to be the ablation zone via a mouse or touchscreen. In one embodiment, the suggested planned ablation zone is automatically generated by the controller, such as via an artificial intelligence or other parameters based on the cardiac procedure. For instance, the type of cardiac procedure is determined by the controller, and one or more suggested planned ablation zones are presented to a user such as via a visualization.

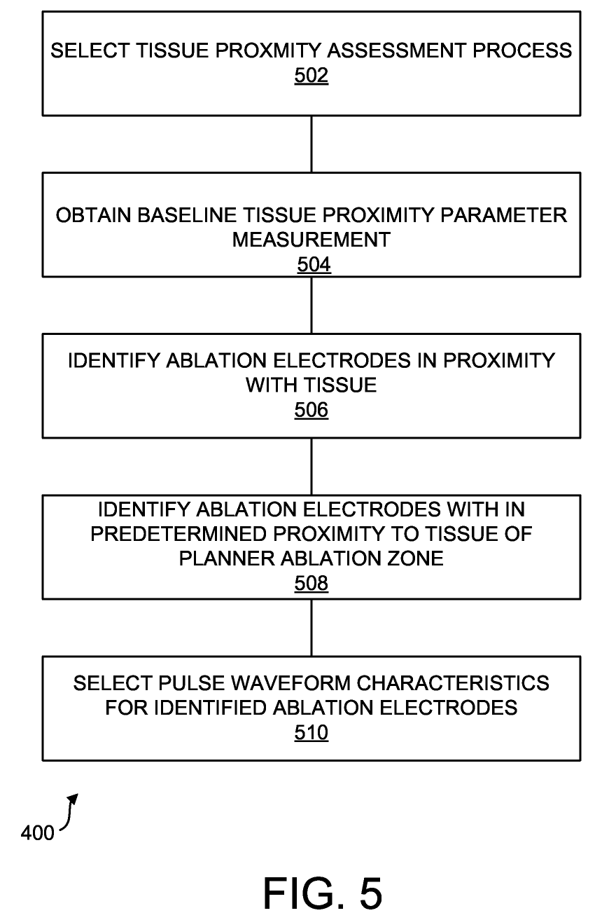

The controller is configured to determine a first subset of ablation electrodes that within a predetermined proximity to the planned ablation zone at 404. In one embodiment, the predetermined proximity is based on whether the position of the ablation electrode with respect to the tissue is close enough to provide an effective lesion resulting from determined pulse parameters. In one embodiment, contact with the tissue is within a predetermined proximity. An activated ablation electrode in contact with tissue will provide an effective lesion. In another example, an activated ablation electrode in a blood pool and not within the predetermined proximity to tissue can cause hemolysis, which can have adverse effects for a patient including acute kidney injury. In one embodiment, the predetermined proximity is a distance. In another embodiment, the predetermined proximity is based on an impedance measurement. For instance, an impedance measurement is indicative of proximity to tissue, and a determination of whether the impedance is indicative of a predetermined proximity is made based on whether the impedance is within a range of impedances. In some embodiments, the first subset of ablation electrodes is fewer than the plurality of ablation electrodes. In some embodiments, the controller is also configured to determine which of the ablation electrodes, if any, are in a second subset of ablation electrodes are not within the predetermined proximity to the planned ablation zone. Exemplary methodologies for identifying the tissue-contacting ablation electrodes are discussed in greater detail in connection with FIG. 5. For example, if an ablation electrode is determined not to be within the predetermined proximity to the cardiac tissue, the ablation electrode is determined to be in the second subset of ablation electrodes. Also, if an ablation electrode is determined to be within the predetermined proximity to the cardiac tissue but that cardiac tissue is not within the predetermined proximity to cardiac tissue corresponding with the planned ablation zone, the ablation electrode is determined to be in the second subset of ablation electrodes. In one example, the program includes logic to determine the first subset of electrodes and the second subset of electrodes. The controller can be configured to select ablation electrodes among the subset of ablation electrodes identified as being in contact with cardiac tissue and within cardiac tissue corresponding to the planned ablation to which the pulsed waveform is to be delivered or applied. In some embodiments, all of the ablation electrodes identified as being within the predetermined proximity to the target tissue and within the planned ablation zone may be selected, or alternatively, fewer than all of the tissue-contacting electrodes within the corresponding planned ablation zone may be selected for pulsed waveform delivery based on predetermined criteria.

In one embodiment, the controller is configured to determine a first subset of ablation electrodes in contact with the cardiac tissue and within the planned ablation zone at 404. In some embodiments, the controller is also configured to determine which of the ablation electrodes, if any, are in a second subset of ablation electrodes are not in contact with the target tissue or not in the planned ablation zone. For example, if an ablation electrode is determined not to be in contact with the cardiac tissue, the ablation electrode is determined to be in the second subset of ablation electrodes. Also, if an ablation electrode is determined to be in contact with the cardiac tissue but that cardiac tissue is not within the planned ablation zone, the ablation electrode is determined to be in the second subset of ablation electrodes.

The controller is configured to adapt a pulsed waveform by selecting one or more characteristics of the pulsed electrical signal are selected based on the configuration, e.g., the number, spacing, orientation etc., of the first subset of ablation electrodes identified as being in contact with target tissue and within the corresponding planned ablation zone. For example, in embodiments, it may be clinically beneficial to spatially distribute the delivery of the pulsed waveform to selected electrodes as widely as possible to enhance the creation of wide-area focal ablation lesions. Accordingly, waveform characteristics such as the sequencing of pulsed waveform delivery to specific selected ablation electrodes (e.g., two or more spline ablation electrodes, or electrodes selected based on a desired active ablation electrode surface area) can be selected to optimize the tissue surface exposed to the ablative electric field created by the pulsed waveform. As an additional example, the overall duration of the pulsed waveform delivery, e.g., as in the number of heartbeats over which the pulsed waveform is delivered, can be dynamically selected by the controller based on the configuration of the selected ablation electrodes. In embodiments, one or more parameters of the pulsed electrical signal can also be adapted by the controller based on the selected ablation electrode configuration. Exemplary parameters may include, without limitation, pulse width, pulse spacing, pulse amplitudes (i.e., current, voltage), number of pulses per delivery, and the like. In still other embodiments, pulsed waveform parameters can be dynamically adjusted during delivery.

In embodiments, the selected ablation electrodes can be configured for delivery of ablative energy in either monopolar or bipolar modes. In one embodiment, the selected ablation electrodes can be configured to operate in a monopolar mode, with an indifferent or return electrode located remotely from the catheter 105, e.g., as with the pad dispersive electrode located on the patient, such as a body-surface electrode. In embodiments, one or more of selected electrodes can be paired with one or more other ablation electrodes of the subset to form a bipolar ablation electrode pair for delivery of ablative PFA energy in a bipolar mode. In still other embodiments, a bipolar ablation electrode pair may be formed by one or more of the selected ablation electrodes and one or more other electrodes located on the catheter 105, e.g., one or more shaft electrodes located proximal to the electrode assembly.

In connection with 404, the controller in some embodiments is configured to present on the anatomical map or otherwise on the display the determined first subset of ablation electrodes. In one embodiment, the controller is configured to present on the anatomical map the ablation electrodes within the predetermined proximity to the planned ablation zone. For instance, the controller is configured to present on the anatomical map the ablation electrodes in contact with tissue corresponding with the planned ablation zone. In one embodiment, the controller can present the first set of ablation electrodes in a color from a plurality of colors, such as green along with the planned ablation zone. In another embodiment, the controller is configured to present on the anatomical map the ablation electrodes in the second subset of ablation electrodes in another color of the plurality of colors such as red. In still another embodiment, the controller can be configured to present an expected lesion based on the expected delivery of ablative energy to the first subset of ablation electrodes. The expected lesion is determined based on the pulse parameters and proximity of the expected activated ablation electrodes and can appear as a mark on the surface of the tissue of the visualization. In some embodiments, the expected lesion can include a depth within the tissue in a three-dimensional representation of the cardiac anatomy. Based on the information presented in the display, a clinician can determine the value of proceeding to activate the first subset of ablation electrodes or whether to maneuver the catheter to another location to include more or less ablation electrodes within the first subset of electrodes. In another embodiment, the controller provides an alert via an output device, such as a speaker with an audio signal or the display with a visualization, whether none or less than a threshold amount of ablation electrodes are in the first subset of ablation electrodes.

A pulsed electrical signal configured for ablation via electroporation is applied to the first subset of ablation electrodes and not to the second subset of ablation electrodes at 406. In embodiments, the pulsed electrical signal configured for electroporation is activated at only those electrodes within the predetermined proximity to the planned ablation zone and not activated in ablation electrodes not within the predetermined proximity to the planned ablation zone. In some embodiments, the pulsed electrical signal configured for electroporation is activated at only those electrodes that are both within the planned ablation zone and in contact with or the cardiac tissue to deliver ablation energy. The pulsed waveform is subsequently applied to only the selected ablation electrodes in accordance with the delivery strategy selected. In some embodiments, one or more pulsed waveform characteristics may additionally be dynamically adjusted by the controller after the initial delivery to the selected electrodes. For example, in an embodiment, an initial tissue impedance may be sensed by the ablation electrodes and/or sensing electrodes on the electrode assembly prior to delivery of the ablative pulse waveform, and subsequently adjusted during the ablative energy delivery, e.g., prior to each heartbeat or pulse packet delivery.

FIG. 5 illustrates a process 500 of configuring a controller, such as controller 300, to determine which of the ablation electrodes of an electroporation catheter, such as catheter 200, are within the predetermined proximity to the target tissue, and to select or activate such ablation electrodes to provide PFA and deselect or deactivate ablation electrodes that are not within the predetermined proximity to the target tissue. The controller 300 can be configured to adapt the PFA waveform provided to the selected ablation electrodes based on the configuration of the selected electrodes. For example, the controller can be implemented as part of the electroporation catheter system 60 or operably coupled to the EAM system 70. Process 500 can be included in an implementation or example of process 400. Process 500 can be use with a catheter including an electrode assembly having a plurality of ablation electrodes, wherein the catheter is adapted to be maneuvered in the chamber of the heart.

In the various embodiments, the system 50 may be configured to employ a range of tissue proximity assessment processes or tissue contact assessment processes. As shown in FIG. 5, at 502 one or more tissue proximity assessment processes and tissue contact assessment processes enabled by the system 50 (see FIG. 1) are selected. In one embodiment, the tissue proximity assessment processes and tissue contact assessment process can be determined based on the received planned ablation zone from 402, the predetermined proximity, and the type of tissue to be ablated. A baseline measurement of one or more tissue proximity parameters corresponding to the selected tissue proximity assessment processes and tissue contact assessment processes such as for calibration at 504. Then at 506, after moving the electrode assembly to a location to which at least some portion of the electrode assembly is in proximity or in contact with the target tissue, the controller 300 utilizes the selected tissue proximity assessment processes or tissue contact assessment to identify a subset of the ablation electrodes that are in proximity or in contact with the target tissue, and then selects those ablation electrodes (or a subset of the tissue in proximity ablation electrodes or tissue-contacting ablation electrodes) for consideration as to whether the ablation electrodes are within the area of tissue represented by the planned ablation zone at 508. In one embodiment, the controller determines which of the ablation electrodes are in the planned ablation zone at 508 and identifies the first and second subsets of ablation electrodes. Additionally, at 510, the controller 300 selects one or more pulsed signal characteristics for delivery to the selected ablation electrodes based on the configuration of the selected ablation electrodes and within the area of tissue represented by the planned ablation zone, as described above in connection with process 400. The pulsed waveform is applied to the first subset of ablation electrodes.

In one embodiment utilizing an impedance-based proximity assessment process or contact assessment process in which impedance between a given ablation electrode and a second electrode (e.g., another ablation electrode, a sensing electrode, or a remote indifferent electrode) is utilized as the tissue proximity parameter, the controller is configured to deliver pre-pulse signals, or pre-pulses to the ablation electrodes and/or sense electrodes at the first location with the electrode assembly in the blood pool and then subsequently at a second location at which the electrode assembly is in contact with the target tissue, measure the responsive impedance at each ablation electrode at each location, and compare the measured impedances to assess which ablation electrodes at the second location are actually in contact with tissue and which remain in the blood pool (or which have only minimal contact with tissue). In some embodiments, the pre-pulses are configured to generate electric fields having a field strength below the threshold field strength sufficient to ablate tissue and prior to the generation of the pulsed waveform for ablation. The pre-pulses are not configured to ablate tissue via electroporation and, in some embodiments, include electrical characteristics such as frequency and current that are outside a threshold to stimulate the cardiac tissue. The pre-pulses can be configured to be generally continuous or intermittent signals used to measure impedance at the ablation electrodes. In one embodiment, if the measured impedance has crossed a predetermined threshold value, such as exceeded the predetermined proximity, the controller 300 will determine that that the ablation electrode is sufficiently within the predetermined proximity. The pre-pulses in some embodiments are applied to detect proximity and are activated when the electrode array is in the cardiac chamber of interest. In one embodiment, the pre-pulse signal for measuring proximity via an impedance determination are distinct from other signals applied to the ablation electrodes prior to the pulsed waveforms for ablation, such as pre-pulses to determine the likelihood of electrical arcing. In one embodiment, a single pre-pulse signal can be applied for more at least one other determination in addition to a determination of impedance.

In exemplary embodiments of the tissue contact assessment process, separate, discrete pre-pulses can be applied to the electrode assembly to determine differences between ablation electrodes and to select the electrodes with the greater current flow. In one example, the impedance or current measurement at an ablation electrode can be compared to the baseline measurement at the ablation electrode to determine whether the electrode is in contact with tissue. In another example, the impedance or current measurement at an ablation electrode can be compared to another ablation electrode in the electrode assembly, such as an ablation electrode opposite the electrode assembly, such as on an opposite or distant spline like ablation electrode 244A to ablation electrodes 244C or 244D. The pre-pulses can be provided in a bipolar mode, which may be more sensitive to local impedance determinations than in monopolar mode with an indifferent electrode applied as a patch. The bipolar mode can generate an electric field with the pre-pulse between two ablation electrodes or between an ablation electrode and a sense electrode. In another example, the pre-pulse can be a continuous electrical signal rather than a separate discrete signal, and changes in impedance or current flow can be tracked to determine ablation electrodes in contact with the target tissue. For example, phase shifts between voltage and current via impedance can be tracked to determine which of the ablation electrodes has come into contact with the target tissue. Again, ablation electrodes and sense electrodes can be employed with continuous pre-pulse signals.

Other processes can be employed to determine which of the ablation electrodes are in contact with the target tissue and which of the ablation electrodes are not in contact with the target tissue as well. For example, voltage measurements between electrodes can be taken as baseline measurement, and changes in voltage measurements can be used to determine deflection or shape changes in the electrode array, which can be used to determine which of the ablation electrodes are in contact with the target tissue and which of the ablation electrodes are not in contact with the target tissue. Force sensing can be employed with other sensors such as directional or navigational sensors or via rotation or bending of the shaft at the distal end. Electrogram sensing such as amplitude, slew rate, and other characteristics can be employed to confirm tissue proximity or tissue contact. In some examples, an electrode array can include temperature sensors on the splines, and changes in temperature from a baseline measurement can be employed to determine which electrodes are and are not in proximity or not in contact with target tissue. Imaging techniques, such as fluoroscopy, EAM system (spline proximity to map shell), and intracardiac echocardiography (ICE) cardiac imaging, via such inputs as heart map data 310, can be employed to determine which ablation electrodes are in proximity or in contact with the tissue. Still other techniques such as a measurement of resistance to fluid flow, via irrigation lumens disposed in each spline, can be employed to determine which ablation electrodes are in contact with target tissue and which ablation electrodes are not in contact with target tissue.

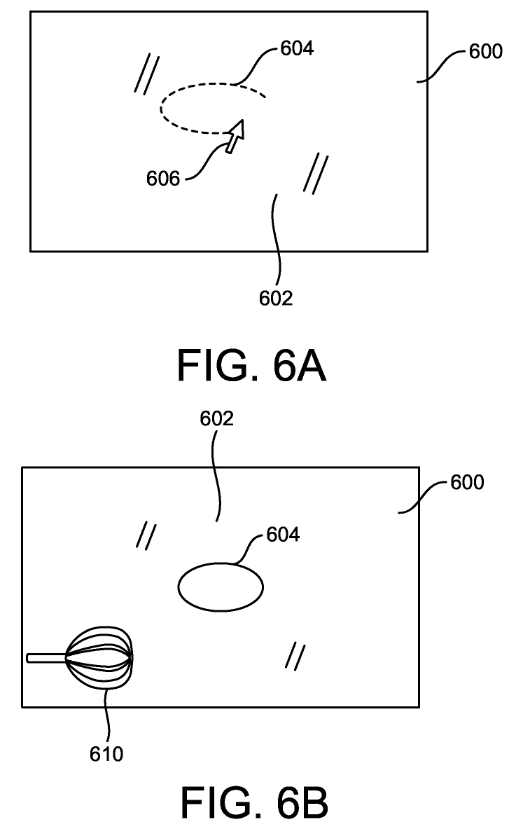

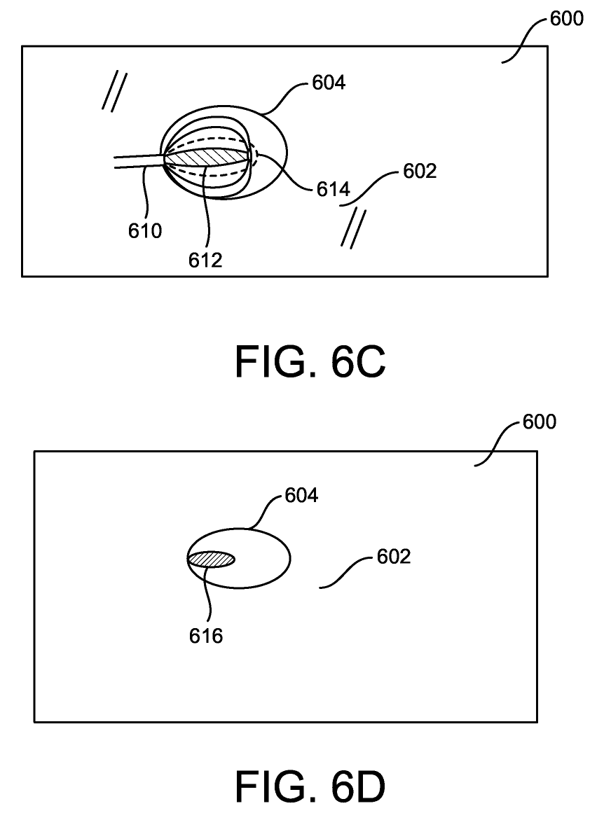

FIGS. 6A-6D illustrates example graphical representations as visualizations generated by the controller 300 implementing the process 400 with electrophysiology system 50 of FIG. 1. FIG. 6A illustrates a graphical representation of visualization 600 of a portion of cardiac tissue 602 of a patient undergoing a cardiac procedure as an anatomical map. In the illustrated embodiment, a user provides a planned ablation zone 604 on the cardiac tissue 602 by manipulating a cursor 606 using an input device. For instance, the cursor 606 is drawing the planned ablation zone 604 onto the surface of the cardiac tissue 602 in the visualization. Once the user is satisfied with the planned ablation zone 604 via pressing an input, such as pressing the return button on a keyboard, for example, the data characterizing the planned ablation zone 604 on an electroanatomical map of a heart is received in the controller. FIG. 6B illustrates a graphical representation of the visualization 600 including the planned ablation zone 604 input into the electroanatomical map and a representation of a distal end of a catheter 610, the representation including the electrode assembly, used in the procedure nearing proximity to tissue corresponding to the planned ablation zone as a clinician maneuvers the catheter into position. FIG. 6C illustrates a graphical representation of the visualization 600 including the planned ablation zone 604 with the representation of the catheter 610 in a position to ablate tissue. The graphical representation includes an indication of the first subset of ablation electrodes 612. In one embodiment, the visualization 600 can also provide an indication of the parameters for a pulse electrical signal to be provided to the first subset of ablation electrodes (and not to the second subset of ablation electrodes), such as for review by the clinician prior to activating the first subset of ablation electrodes. In the illustrated example, the visualization also includes an expect lesion zone 614 that corresponds with a controller-determined lesion to be formed from activating the first subset of ablation electrodes and not the second subset of electrodes. FIG. 6D illustrates a graphical representation of the visualization 600 after the first subset of ablation electrodes have been activated while in the position indicated in FIG. 6C. The visualization can include the cardiac tissue 602, the planned ablation zone 604, and an ablation tag 616 indicating the lesion formed from activating the first subset of ablation electrodes.

The controller can be configured to apply a pulsed electrical signal to only the selected subset of ablation electrodes (i.e., some or all of the electrodes identified as being in contact with tissue and within the area of tissue corresponding with the planned ablation zone) and not to the remainder of the ablation electrodes. In one example, the pulsed signal to effect ablation is applied in a monopolar mode, and the controller can be configured to apply the pulsed electrical signal to one or more electrodes in contact with the target tissue and within the area of tissue corresponding with the planned ablation zone. In one example of the pulse electrical signal to effect ablation in bipolar mode, a minimum of two ablation electrodes are activated to generate the electrical field. The remainder of the electrodes, i.e., the electrodes not in contact with the tissue, are not used to generate electrical fields for ablation.

In some embodiments, the configuration of the ablation electrodes in contact with the target tissue is used to select characteristics of the pulsed electrical signal, such as the strength, duration, waveform, and other characteristics, is selected based on the configuration. The characteristics of the pulsed electrical signal applied to the ablation electrodes in contact with the target tissue can be based on an output from a lookup table. The remaining electrodes, or electrodes not in contact with the target tissue are not activated. In one example, the output can be based on features such as the ablation electrodes in contact with the tissue, the spacing of the ablation electrodes in the electrode array, whether the ablation is configured in a monopolar mode or a bipolar mode, and other information determined with the pre-pulse electrical signal or determination of which electrodes are in contact, such as impedance or current measurements, which can provide information on the robustness of contact. The waveform applied to the ablation electrodes in contact with the target tissue can be provided as a single preset or adapted into firing pairs. The ablation electrodes in contact with the target tissue can be determined between each application of the pulsed signal. Further, impedance or current measurements can be taken at the ablation electrodes in contact with the target tissue to determine the pulsed electrical signal to apply to the ablation electrodes in contact with the target tissue.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

I claim:

1. A system to perform electroporation ablation of a tissue in a chamber of a patient’s heart, the system comprising:
a catheter including an electrode assembly having a plurality of ablation electrodes, wherein the catheter is adapted to position the electrode assembly in proximity with the tissue; and
a controller configured to:
receive a planned ablation zone on an electroanatomical map of the heart, the planned ablation zone representative of a target tissue on a surface of the tissue intended for ablation;
identify, from among the plurality of ablation electrodes, a first subset of ablation electrodes that are within a predetermined proximity to the planned ablation zone, and a second subset of ablation electrodes that are not within the predetermined proximity to the planned ablation zone; and
apply a pulsed electrical signal to the first subset of ablation electrodes and not to the second subset of ablation electrodes.

2. The system of claim 1, wherein the planned ablation zone is received via user input.

3. The system of claim 2, wherein the planned ablation zone is user-selected from an automatically generated suggested planned ablation zone.

4. The system of claim 3, wherein the suggested planned ablation zone is generated based on an identified cardiac procedure.

5. The system of claim 1, wherein the controller is configured to determine ablation electrodes in contact with the tissue and ablation electrodes within the predetermined proximity to the planned ablation zone.

6. The system of claim 5, wherein the controller is configured to determine ablation electrodes determined in contact with the tissue within the planned ablation zone.

7. The system of claim 1, wherein the controller is configured to determine electrodes within the predetermined proximity to the tissue via an impedance measurement.

8. The system of claim 7, wherein the controller is configured to determine the impedance measurement via a pre-pulse signal.

9. The system of claim 8, wherein the controller is configured to apply the pre-pulse signal to the ablation electrodes only.

10. The system of claim 1, wherein the controller is further configured to generate a visualization of the planned ablation zone with respect to the tissue.

11. The system of claim 10, wherein the controller is configured to generate a representation of the electrode assembly of the catheter with respect to the planned ablation zone.

12. The system of claim 11, wherein the controller is configured to generate a visualization identifying the first subset of ablation electrodes.

13. The system of claim 12, wherein the visualization further identifies the second subset of ablation electrodes.

14. The system of claim 1, wherein the first subset of ablation electrodes includes only ablation electrodes in contact with the target tissue and within the planned ablation zone and the second subset of ablation electrodes includes ablation electrodes not in contact with the tissue or ablation electrodes not within the planned ablation zone.

15. The system of claim 1, wherein the controller is further is configured to adjust ablation parameters based on the identified first subset of ablation electrodes.

16. A system to perform electroporation ablation of a tissue in a chamber of a patient’s heart, the system comprising:
a catheter including an electrode assembly having a plurality of ablation electrodes, wherein the catheter is adapted to position the electrode assembly in proximity with the tissue; and
a controller configured to:
receive a planned ablation zone on an electroanatomical map of the heart, the planned ablation zone representative of a target tissue on a surface of the tissue intended for ablation;
identify, from among the plurality of ablation electrodes, a first subset of ablation electrodes that are in contact with the target tissue within a predetermined proximity to the planned ablation zone, and a second subset of ablation electrodes that are not in contact with the target tissue within the predetermined proximity to the planned ablation zone;
apply a pulsed electrical signal to the first subset of ablation electrodes and not to the second subset of ablation electrodes; and
generate a visualization of the planned ablation zone with respect to the tissue.

17. The system of claim 16, wherein the controller is configured to identify the first and second subsets of ablation electrodes via a pre-pulse signal.

18. The system of claim 17, wherein the controller is configured to determine electrodes in contact with the tissue via an impedance measurement.

19. A method of performing electroporation ablation of a tissue in a chamber of a patient’s heart, comprising a catheter including an electrode assembly having a plurality of ablation electrodes, wherein the catheter is adapted to position the electrode assembly in proximity with the tissue, the method comprising:
receiving a planned ablation zone on an electroanatomical map of the heart, the planned ablation zone representative of a target tissue on a surface of the tissue intended for ablation;
identifying, from among the plurality of ablation electrodes, a first subset of ablation electrodes that are within a predetermined proximity to the planned ablation zone, and a second subset of ablation electrodes that are not within the predetermined proximity to the planned ablation zone; and
applying a pulsed electrical signal to the first subset of ablation electrodes and not to the second subset of ablation electrodes.

20. The method of claim 19, wherein the first set of ablation electrodes that are within the predetermined proximity to the planned ablation zone include ablation electrodes that are in contact target tissue and within the planned ablation zone.
